(19) United States
(12) Patent Application Publication
(10) Pub. No.: US 2023/0371889 A1
(43) Pub. Date: Nov. 23, 2023

(54) SPEECH PROCESSING METHOD FOR IDENTIFYING DATA REPRESENTATIONS FOR USE IN MONITORING OR DIAGNOSIS OF A HEALTH CONDITION

(71) Applicant: Novoic Ltd.

(72) Inventors: Jack Weston, Greater London (GB); Emil Fristed, London (GB)

(21) Appl. No.: 18/366,145

(22) Filed: Aug. 7, 2023

Related U.S. Application Data
(63) Continuation of application No. PCT/EP2022/051453, filed on Jan. 24, 2022.

(30) Foreign Application Priority Data
Feb. 5, 2021 (EP) 21155635.2

(51) Int. Cl. A61B 5/00; G16H 50/20; G06N 3/08; G10L 25/66; G10L 25/30; G10L 17/02; G10L 17/04

(52) U.S. Cl. CPC A61B 5/4803; G16H 50/20; G06N 3/08; G10L 25/66; G10L 25/30; G10L 17/02; G10L 17/04; A61B 5/7267; A61B 5/7275; A61B 5/4088; A61B 5/4064

(57) ABSTRACT

The invention relates to a computer-implemented method for identifying clinically meaningful representations of speech data for monitoring or diagnosis of a health condition, the method comprising: providing a main model comprising a trained neural network, trained to map an input representation encoding input speech data from a speaker to an output representation for use in providing a health condition prediction, the neural network comprising one or more internal network layers each comprising an internal representation which is passed to a subsequent network layer; inputting speech data from a speaker into the main model to form the internal representations of the input speech data; training a probe comprising a machine learning model, independently to the training of the main model, to map an internal representation of the input speech data an internal network layer of the main model to an independently determined measure of a clinically relevant feature of the input speech data or the speaker, where a clinically relevant feature is a property of the input speech or speaker that is impacted by a health condition. By training a probe externally to the main model, to map an internal representation to an independently determined measure of a clinically relevant feature, it is possible to identify associations within the internal representations that otherwise might not be found by the main model and to build improved representations based on these associations.

### CROSS-REFERENCE TO RELATED APPLICATIONS

This application is a bypass continuation of International Application No. PCT/EP2022/051453, filed on Jan. 24, 2022, which in turn claims priority to European Application No. 21155635.2, filed on Feb. 5, 2021. Each of these applications is incorporated herein by reference in its entirety for all purposes.

TECHNICAL HELD The present invention relates to a method for processing speech data to determine clinically meaningful data representations for use in speech analysis tasks for the monitoring or diagnosis of a health condition.

### BACKGROUND

The rapid pace of development in the field of machine learning, together with increasing computing power and the availability of large clinical data sets, has led to the increasing application of computational methods in the analysis, interpretation, and comprehension of medical and healthcare data. The potential for machine learning to transform the healthcare industry is widely recognised and is increasingly looked to as a potential solution to increasing pressures faced in healthcare due to growing ageing populations.

Applications of artificial intelligence in healthcare include the use of machine learning to predict the pharmaceutical properties of molecular compounds and targets for drug discovery, pattern recognition and segmentations techniques on medical images to enable faster diagnosis and tracking of disease progression, and developing deep-learning techniques on multimodal data sources such as combining genomic and clinical data to develop new predictive models.

More recent developments have attempted to analyse spoken language to extract clinically meaningful information. This has involved both text and character-based analysis of the linguistic component of speech (e.g. semantics, grammar, syntax, conversational analysis), and audio-based analysis of the acoustic components of speech (e.g. prosody and wave-level abnormalities associated with vocal cord functioning). Generally disparate methodologies have been required for analysing these two components.

Partly driven by developments in audio processing and natural language processing, machine learning algorithms have been applied to identify acoustic and linguistic impairments in spoken language indicative of neurodegenerative disorders, such as Alzheimer's disease. Models are generally trained to do one of two things: classification (e.g. based on their speech, decide if someone has mild cognitive impairment or not) or regression (e.g. based on their speech, predict a severity score of cognitive impairment, for example a Mini-Mental State Exam—MMSE—score). Despite significant progress in these approaches, there remain a number of significant challenges.

One such challenge is how to reduce the dimensionality of the complex input speech data in a way which retains the important clinical information and allows the model to effectively learn the required associations. One common way of doing this is using “features” extracted from speech as input. Features can be audio or text based, for example the noun rate in someone's speech, or the frequency of pauses.

Extracting features from the input data significantly reduces the dimensionality of the input data, with the intention of making it easier for a model to learn associations within the data. This technique also allows features with known clinical rationale to be used such that, for example, the model doesn't have to learn how to calculate the noun rate from a free speech sample, and that the noun rate matters for early Alzheimer's, but rather it can just learn the association between the already extracted noun rate and early Alzheimer's.

However, there are certain drawbacks to using feature extraction to represent the input data. Firstly, selecting the features manually could result in clinical information within the input speech being lost. There may be associations between parts of the input data and a particular health condition which are not known and are lost when encoding the input speech data into the selected feature space. Although using known clinical understanding to help select features can be useful in imbuing models with knowledge of a health condition, it could also potentially shape the model to have an overdependence on current knowledge of a disease and restrict the model in finding new associations.

An alternative approach to feature extraction, which has significant success in non-clinical applications of machine learning to natural language processing, is representation learning. Rather than selecting and extracting features of the input speech data in advance, representation learning models independently learn to find the most important associations within the input data. This gives the model more flexibility to identify associations in the input data that were previously unknown to potentially move beyond the above-described restrictions of feature-based models to achieve more accurate results. The applicants have described the application of a new representation learning model to the monitoring or diagnosis of a health condition in European Patent Application number 20185364.2.

One of the drawback of representation learning models is that they must be trained on very large data sets and the required clinical data is very limited. In the above application, the applicants proposed a solution to this problem with a new model and training strategy involving first training on large unlabelled data sets before fine tuning on more limited clinical data. However the limited clinical data remains a challenge and there is an effort to improve representation learning models so that they can effectively find the associations in these limited data sets.

A further problem, common to both approaches but particularly a challenge for representation learning models, is explainability. Deep learning models are often considered “black boxes” where the relationship between the input data and prediction is difficult to probe and understand. This is a particular problem for the use of such algorithms in clinical applications, such as the analysis of speech to predict Alzheimer's, where understanding which changes in speech are predictive of a particular condition is important to give the necessary confidence in using the models in a clinical setting—and to further understanding of a particular disease.

Accordingly there is a need for a new approach to the application of machine learning to clinical monitoring and diagnosis that makes progress in overcoming the problems described above. In particular, there is a need for an improved method of identifying relevant features in speech data which are predictive of certain health conditions. It is particularly desirable to find a method of identifying associations within speech data which might otherwise not be identified by current state of the art representation learning models and to achieve efficacy on the limited clinical data sets available. A related aim is improving explainability so that clinicians can understand why a model is making a certain prediction and this information can be used to further understanding of a disease and to improve models for monitoring and diagnosing those diseases.

### SUMMARY

According to a first aspect of the invention there is provided computer-implemented method for identifying clinically meaningful representations of speech data for monitoring or diagnosis of a health condition, the method comprising: providing a main model comprising a trained neural network, trained to map an input representation encoding input speech data from a speaker to an output representation for use in providing a health condition prediction, the neural network comprising one or more internal network layers each comprising an internal representation which is passed to a subsequent network layer; inputting speech data from a speaker into the main model to form the internal representations of the input speech data; training a probe comprising a machine learning model, independently to the training of the main model, to map an internal representation of the input speech data an internal network layer of the main model to an independently determined measure of a clinically relevant feature of the input speech data or the speaker, where a clinically relevant feature is a property of the input speech or speaker that is impacted by a health condition.

The first aspect of the invention may be defined alternatively as a computer-implemented method for identifying representations of speech data for monitoring or diagnosis of a health condition, the method comprising: providing a main model comprising a trained neural network, trained to map an input representation encoding input speech data from a speaker to an output representation for use in providing a health condition prediction, the neural network comprising one or more internal network layers each comprising a representation of the input speech data which is passed to a subsequent network layer, where the representations of the internal network layers are referred to as internal representations of the trained neural network; inputting speech data from a speaker into the main model to form the internal representations of the input speech data; training a probe comprising a machine learning model, independently to the training of the main model, to map an internal representation of the input speech data to a measure of a clinically relevant feature of the input speech data or the speaker, where a clinically relevant feature is a property of the input speech or speaker that is impacted by a health condition.

By training a probe externally to the main model, to map an internal representation to a measure of a clinically relevant feature, it is possible to identify associations within the internal representations that otherwise might not be found by the main model. Given the limited data available for training a clinically predictive main model, there is a high likelihood that the main model will not find all of the clinically relevant associations in the input data. By selecting certain clinically relevant features known to be relevant to a particular condition and mapping internal representations to these features, specific parts of the internal representation which are linked to those features can be identified which have not been found by the main model. These elements of the representation can then be used as features to represent speech data input into the main model or a new model to improve performance in monitoring or diagnosis of a health condition.

The method therefore retains the benefits of representation learning models in not placing any restrictions on the main model in identifying associations in the speech data but also allows for clinical knowledge to be utilised by the probe model to identify further features, effectively achieving advantages of both feature-based and representation learning approaches to significantly improve the identification of clinically meaningful representations usable as biomarkers for health conditions.

The method also improves explainability as it allows the internal representations of the model within the “black box”—to be probed to understand their association with the health condition prediction. In particular, the probe model can be used to analyse the internal representations to understand the clinically relevant information which is being used by the main model to make a prediction—and to confirm that clinically relevant information is being successfully transformed through the network and the model is not exploiting an unknown or undesired asymmetry in the data.

Furthermore, the method can provide a quantified measure of the amount of relevant information encoded in the representations by providing an objective measure of the amount of training data, or the size of model, required to give a certain level of prediction accuracy.

As well as improving confidence in the model, the method can provide an improved, more accurate diagnosis by using the explanatory variables analysed by the probe as well as the main model prediction. Understanding why the main model is making a prediction also further develops understanding of a particular disease or health condition and allows models to be improved. The method therefore makes significant progress in improving explainability—essential for the more widespread implementation of machine learning based techniques in a clinical setting and for furthering the understanding of a particular health condition.

Training the probe “independently to the training of the main model” means the probe is trained in a separate training task. Alternatively stated, the probe is not trained together with the main model. In particular, the probe is not trained with a combined training objective to that used in training the main model. In other words, the probe is trained separately to the training of the main model. In preferable examples, training the probe independently of the main model comprises fixing the main model after training and, in a separate training task, training the probe to map a fixed internal representation of the input speech data to the independently determined measure of a clinically relevant feature of the input speech data or the speaker. In this way, the representations of the trained main model, including the internal representations, are fixed. The probe is then takes a fixed internal representation of the speech data from the main model as input is trained to map this representation to the measure of a clinically relevant feature.

Preferably the probe is trained to map an internal representation of the input speech data an internal network layer of the main model to an independently determined measure of a clinically relevant feature of the input speech data or the speaker. An “independently determined” measure of a clinically relevant feature means that the measure of the feature determined independently of the main model, i.e. a measure of the clinically relevant feature not used as an input or output main model determined by the main model and is not used in training of the main model. Preferably it means that it is a measure of a property of the speech or speaker, such as an objective property of the speech of a subjective assessment by a clinician, further examples of which are provided below.

Preferably the computer-implemented method is usable for identifying clinically meaningful representations. As described herein, the term “clinically meaningful representations” means representations suitable for use in the monitoring or diagnosis of a health condition.

As used herein the term “internal representation” refers to the representation of an internal network layer of the neural network. As described herein an internal network layer of the neural network comprises a network layer which has a subsequent network layer within the neural network to which it passes information. Preferably the neural network comprises an input layer, an output layer and one or more internal network layers between the input layer and output layer. Each layer comprises a representation of the input speech data that differs from the other network layers. In particular, the input speech data is encoded in the representation of each network layer as it passed through the network. During training of the neural network the representations change in an effort to transmit information through the network such that the representation of the output layer is usable for providing a health condition, i.e. in line with the training goal. The layers and reach a final state, i.e. having a final form of their representation, upon completion of training. The method comprises mapping a data representation of the input speech from an internal network layer to the measure of a clinically meaningful feature.

Preferably the method further comprises using the trained probe to: confirm that the internal representation of the main model contains information associated with the clinically relevant feature. In particular, by confirming that the probe can predict a clinically relevant feature using the internal representation it can be determined that the representation encodes related information. Similarly by comparing prediction accuracy at different layers of the model it can be confirmed that clinically relevant speech information is being transferred through the model. The method can be implemented in a clinical setting to confirm that a diagnostic tool implementing a machine learning model is working correctly in identifying relevant information within speech.

Preferably the method comprises: training a probe to map an internal representation of a first internal network layer of the main model to an independently determined measure of a clinically relevant feature; and training a probe to map an internal representation of a second internal network layer of the main model to the independently determined measure of a clinically relevant feature; such that, by comparing the ability of the trained probe to predict the clinically relevant feature using internal representations at different network layers, the efficacy of the main model in identifying clinically relevant representations can be determined. In particular, by comparing the prediction accuracy of a probe model, or the information required by the model/size of the probe model required to provide a given level of prediction accuracy, at different layers of a network it can be determined that a trained machine learning model is functioning correctly in transferring clinically relevant information through the network. This can be used to determine the confidence of a health condition prediction and ensure the model is not using unintended patterns in the data or nuisance variables to make predictions.

Preferably the method comprises using the trained probe model to identify elements of the internal representation that: encode more information usable by the probe for predicting the clinically relevant feature relative to the remaining elements of the representation or other internal representations; and/or decouple from the remaining elements of the internal representation in predicting the clinically relevant feature. In this way, the method can be used to determine parts of the representation that are particularly associated with a particular clinically relevant feature of the speech or speaker. This provides the possibility of forming new representations based on those elements that are particularly related to a particular clinical feature and discarding elements that do not aid in making a prediction. The method therefore allows for new representations to be formed which are provide stronger predictions of certain health condition affected properties of speech. The term “decouple” is intended to refer to the situation in which the representation has elements which are strongly predictive and therefore strongly linked to a particular condition and elements which are only weakly predictive or non-predictive, meaning the representation can be decoupled into predictive elements and non-predictive elements and just the predictive elements can be selected.

Alternatively elements of the internal representation may be selected which provide a prediction of a given accuracy with the least amount of training data to be used when training the model or with the smallest or simplest structure of probe model. These are both indications that more predictive information is encoded in these elements of the representation than others.

Preferably elements of the representations and/or layers within the main model are identified by training one or more probe models to map representations to a clinically relevant features and selecting elements according to one or more of: (1) the accuracy of the prediction of the clinically relevant feature provided by the elements or the representations of a particular layer; (2) the degree to which certain elements of the layer decouple from remaining elements of the layer in making the prediction; (3) the size or complexity of the probe model required to provide a given prediction accuracy, (4) the amount of input speech data needed to train the probe to achieve a given prediction accuracy; (5) the minimum amount of data per example to perform the task; (6) “cross-entropy loss of the model”.

Preferably the model is trained to identify predictive elements, representations or layers using a “codelength” which reflects the model size and the amount of information required to pass for a task, given that the model is known. It means that in terms of a code, the ability of a probe to achieve good quality using small amount of data or using a small probe architecture reflect the same property: the strength of the regularity in the data.

Preferably the elements of the internal representation are identified according to parameters of the machine learning model of the probe learnt during training, wherein the parameters preferably comprise one or more of weights, biases and activations learnt by the machine learning model of the probe. The learnt parameters of the probe provide an indication of the elements of the representation which are most significant as the probe will learn to apply increasingly weight to those elements of the representation which are most strongly predictive.

The method preferably further comprises: using the identified elements of the internal representation to form representations of input speech data usable for providing a health condition prediction associated with the clinically relevant feature. In particular the identified elements may be combined into a new representation for encoding input speech data to provide a health condition prediction. This provides a way of building stronger representations for use as biomarkers. In particular it allows for additional external information to be utilised in the constructing of the new representations. For example, by using a probe model to identify elements of the representation learned by the main model that predict the speaker's score on a neuropsychological test and combining these in a new representation, the new representation combines information learnt by the original probe model and external information from the independent test to form stronger representations.

Preferably the elements identified by the probe are used to form speech data representations which are invariant to one or more of: speaker identity, speaker age, speaker gender. For example a probe model may be used to select elements which are non-predictive of speaker identity. For example the probe may be configured to map a representation to features associated with timbre of the speech, which is characteristic of the speaker. Elements may be selected which are least predictive of the characteristics components of speech. By forming new representations from these elements, the new representations may be substantially de-identified from the speaker. Similarly, a probe may be used to map the elements of a representation to the non-characteristic components of speech, for example non-timbral prosody components such as rhythm, tempo and pitch features and the elements which are most strongly predictive of these features may be selected to provide de-identified representations.

Preferably, where the main model has a plurality of internal network layers, the method comprises: training a probe for each of a plurality of the internal network layers to map the corresponding internal representation to the measure of the clinically relevant feature of the input speech data; selecting one or more layers according to one or more of: (1) the accuracy of the prediction of the clinically relevant feature provided by the internal representation of the layer; (2) the degree to which certain elements of the layer decouple from remaining elements of the layer in making the prediction; (3) the size or complexity of the probe model required to provide a given prediction accuracy, and (4) the amount of input speech data needed to train the probe to achieve a given prediction accuracy. These measures all provide means to assess the extent to which the elements of the representation encode information which is predictive of the clinically relevant feature. The third and fourth options are particularly advantageous as they provide a robust method for providing a quantitative measure of information encoded within the representation. In particular, the method preferably comprises information-theoretic probing with minimum description length (MDL), in which the probe is trained to effectively transmit the required data. Therefore, the measure of interest changes from probe accuracy to the description length of labels given representations. In addition to probe quality, the description length evaluates ‘the amount of effort’ needed to achieve the quality

In the variational variant of the above we make a ‘codelength’ (MDL) which is the model size required and the amount of information we need to pass for a task, given that the model is known. It means that in terms of a code, the ability of a probe to achieve good quality using small amount of data or using a small probe architecture reflect the same property: the strength of the regularity in the data. This can preferably be quantified using the loss for the model, preferably using cross-entropy loss.

The main model preferably comprises a supervised, unsupervised, self-supervised or semi-supervised model for making a health condition prediction, the method further comprising: inputting the identified elements of the internal representation into a machine learning model to determine a prediction of the health condition based solely on the identified elements associated with the clinically relevant feature.

The main model may preferably comprises an unsupervised or self-supervised model as described in European application number 20185364.5.

The probe preferably comprises a linear model, multi-layer perceptron, an attention-based model or a Bayesian neural network. These models provide the required function of providing a prediction based on an input representation with a relatively simple structure.

The method preferably comprises: fixing the main model once trained; and subsequently training the probe model to map an internal representation of an internal network layer of the fixed main model to the independently determined measure of a clinically relevant feature. In this way, the representations of the main model are fixed and cannot change during training of the probe.

Training the probe preferably comprises: performing disentanglement on the internal representation of an internal network layer to provide a disentangled internal representation; training the machine learning model of the probe to map the disentangled internal representation to the independently determined measure of a clinically relevant feature. Providing an intermediate disentanglement step provides a number of advantages. In some situations the elements of the representation may not sufficiently decouple so that a sub-selection can be made of elements which are strongly predictive. By applying a disentanglement step on the internal representations the elements of the representation may be transposed to a new vector space in which they are de-coupled prior to application of the probe. This allows the probe to select disentangled elements of the representation which are more strongly decoupled than would otherwise be possible. In these examples the probe model is considered to encompass an initial disentanglement module such that training the probe to map internal representations of the main model comprises performing disentanglement on the internal representations and then training the machine learning model of the probe to map the disentangled representations to the clinically relevant feature.

Performing disentanglement on the internal representation preferably comprises performing a principal component analysis.

Preferably the clinically relevant feature comprises one or more of: an objective property of the input speech, preferably a phonological, prosodic, lexico-semantic or syntactic property; a property of the speaker, preferably the speaker's score on a neuropsychological test; a clinician's rating of the speech or speaker. In certain preferably examples the clinically relevant feature may comprise any feature of the speech of speaker which is impacted by a health condition other than an objective property of the language used. In particular preferably the clinically relevant feature comprises one or more of: a non-linguistic property of the speech, an acoustic property of the speech, a phonological property of the speech, prosodic property of the speech, a property of the speaker, the speaker's score on a neuropsychological test; a clinician's rating of the speech or speaker. Using these kind of non-linguistic/non-lexico-semantic properties of speech is particularly advantageous. In particular, there is a greater chance that the main model will miss patterns in this kind of data. It also provides a way of bringing in external information into the model, for example through assessment of the speaker, rather than relying on objective properties of the linguistic/syntactic properties of the speech which are clearly accessible by the model. Furthermore, this type of information is particularly crucial in assessing cognitive impairment and is information which is less readily interpretable by conventional methods of cognitive impairment assessment and more likely to be missed in assessment of a patient.

Preferably the main model comprises a supervised, unsupervised, self-supervised or semi-supervised machine learning model, preferably a classifier or regression model, trained to map the input representation to an output associated with a health condition. Preferably the main model is trained using unsupervised (specifically self-supervised) learning, for example by training the model to predict a feature or property of the input. Preferably the main model is trained using a masking objective by masking a components of the input and training the model to predict the masked component, optionally using a contrastive loss in which the model is trained to select between a fixed number of possible options for the masked input.

Preferably the main model is configured to take audio data as input, as either raw audio or audio representations. Preferably the target output is also audio, for example audio representations. Preferably the main model is a prosody encoder trained to map input audio to prosodic representations. Preferably the probe model is trained to map an audio representation or a prosodic representations to a clinically relevant feature. Preferably the clinically relevant feature is representative of a component of prosody, for example one or more of timbre, pitch, rhythm, tempo. For pitch a probe model may be trained to predict the median pitch. For rhythm probe models may be trained to predict median word intensity and number of syllables. For tempo, probe models may be trained to predict articulation rate (syllables per second), speech rate, average syllable duration, and word duration (including pre-silence). For timbre, probe models may be trained to predict the median formants F1, F2, F3 (shifted).

Preferably providing the trained main model comprises: pre-training the main model, preferably using an unsupervised learning task on an unlabelled training data set; performing task specific training on the pre-trained main model using a second training data set with labels associated with a specific health monitoring or diagnosis task, to provide the trained main model. In this way, pre-training can be carried out on large widely available unlabelled general purpose data sets and more limited health related data sets are only required for a subsequent task-specific training step to optimise the model for a particular speech processing task. This allows for significant performance gains despite limited specific data.

Preferably training the main model comprises training using training data comprising audio speech data, the method comprising: obtaining one or more linguistic representations that each encode a sub-word, word, or multiple word sequence, of the audio speech data; obtaining one or more audio representations that each encode audio content of a segment of the audio speech data; combining the linguistic representations and audio representations into an input sequence comprising: linguistic representations of a sequence of one or more words or sub-words of the audio speech data; and audio representations of segments of the audio speech data, where the segments together contain the sequence of the one or more words or sub-words; the method further comprising: training a machine learning model using unsupervised learning to map the input sequence to a target output to learn combined audio-linguistic representations of the audio speech data for use in speech analysis for monitoring or diagnosis of a health condition.

Preferably initial audio representations may be formed by pre-processing the audio speech data to remove timbral information; encoding sections of the pre-processed audio speech data into audio representations by inputting sections of the pre-processed audio data into a prosody encoder, the prosody encoder comprising a machine learning model trained using self-supervised learning to map sections of the pre-processed audio data to corresponding audio representations.

Preferably the main model is trained using a loss function configured so as to encourage the model to learn disentangled internal representations. This may be used as an alternative to building in a disentanglement step before applying the probe to the disentangle representations to provide the same advantages in terms of better disentangled representations to apply the probe to.

The main model preferably comprises a classifier or regression model trained to provide a health condition prediction based on the input representation of the input speech data, the method comprising: obtaining a measure of a plurality of clinically relevant features, each clinically relevant feature comprising a property of the speech or speaker which is impacted by the health condition predicted by the main model; and for each clinically relevant feature: applying a separate probe to each of a plurality of the internal network layers of the main model, and training all probes independently to map the corresponding internal representation to the measure of the clinically relevant feature; identifying one or more network layers according to one or more of: (1) the accuracy of the prediction of the clinically relevant feature provided by the internal representation of the layer; (2) the degree to which certain elements of the layer decouple from remaining elements of the layer in making the prediction; (3) the size or complexity of the probe model required to provide a given prediction accuracy, and (4) the amount of input speech data needed to train the probe to achieve a given prediction accuracy; selecting elements of the corresponding internal representations that encode more information usable by the probe for predicting the clinically relevant feature relative to the remaining elements of the representation or other internal representations; and/or decouple from the remaining elements of the internal representation in predicting the clinically relevant feature; combining the selected elements into one or more vectors.

Preferably the one or more vectors are invariant to one or more nuisance variables, the nuisance variables preferably comprising one or more of: speaker gender, age or identity. In particular, preferably the models are trained to identify elements which are non-identifying of the speaker. For example a probe may be trained to find elements which are least predictive for speaker-characteristic elements of speech.

Preferably the method further comprises encoding input speech data into the one or more vectors; inputting the vectors into the main model or another machine learning model to provide a health condition prediction. In this way, speech data may be encoded in the stronger representations built using the methods of the present invention and used to train a predictive model to make a more accurate health condition prediction.

The health condition may be related to one or more of a cognitive or neurodegenerative disease, motor disorder, affective disorder, neurobehavioral condition, head injury or stroke.

Although the above aspects of the invention define probing as mapping an internal representation to a clinically relevant feature, in some examples of the present invention the method may comprise mapping an output representation or a representation in a final layer to a clinically relevant feature.

For explainability purposes, we may wish to measure how well a feature is represented in a given representation. We use the prequential (or online) approach to minimum description length (MDL) to quantify the regularity between representations and labels. Formally, MDL measures the number of bits required to transmit the labels given the representations. If a feature is highly extractable from a given representation, a model trained to detect said feature will converge quickly, resulting in a small MDL. Computing the MDL using the prequential approach requires sequential training and evaluation. We partition the train set into timesteps and train our probe such that at timestep to calculate the codelength as per the standard prequential method.

### BRIEF DESCRIPTION OF THE DRAWINGS

FIG. 1 schematically illustrates a main model comprising a trained neural network for making a health condition prediction;

FIG. 2 schematically illustrates the application of a probe model to the main model of FIG. 1 according to a method of the present invention;

FIG. 3 schematically illustrates the application of multiple probe models to the main model of FIG. 1 according to a method of the present invention;

FIG. 4 illustrates the identified components of the internal representations of the main model according to a method of the present invention;

FIG. 5 illustrates a method of using new representations formed from the identified components of the internal representations of the main model to make a health condition prediction according to a method of the present invention;

FIG. 6 illustrates possible outputs of the methods according to the present invention including components of a health condition prediction relating to each clinically relevant feature probed;

FIG. 7 illustrates a further method according to the present invention which uses a disentangling step prior to application of the probe model;

FIGS. 8A and 8B illustrate a further example of a main model, comprising a prosody encoder, to which the probe of the present invention can be applied

### DETAILED DESCRIPTION

### Overview

The invention relates to a method for probing the internal network layers of a trained clinical predictive model to obtain additional information on why the network is making a health condition prediction and to identify new data representations for encoding speech data, not found by the model, which comprise further clinically relevant information, usable as biomarkers for monitoring or diagnosis of a health condition.

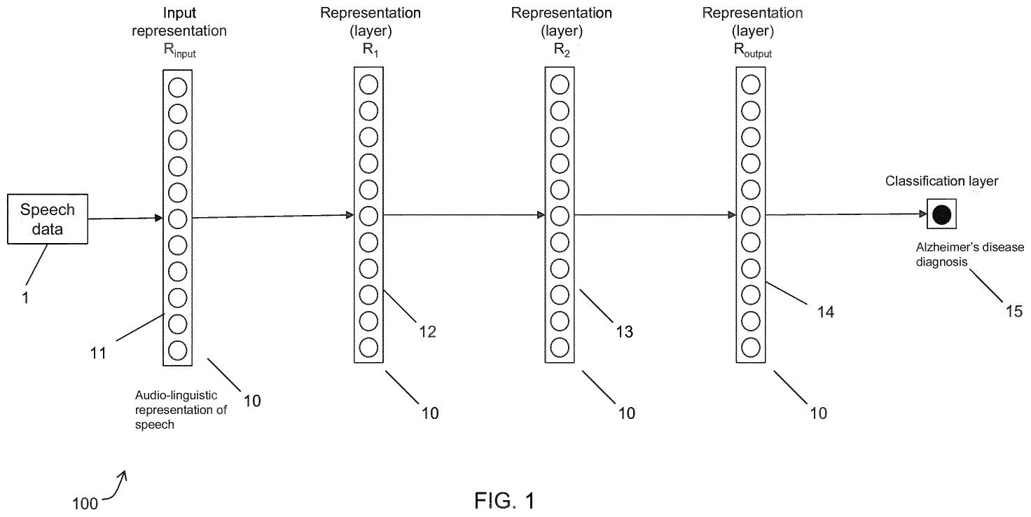

FIG. 1 schematically illustrates a trained main model 100 for making a health condition prediction, which is usable within the present invention. The main model may take various forms and may be trained using various training strategies, as will be described below. In general terms, the main model is a machine learning model for making a health condition prediction based on input speech data and comprises a neural network with a plurality of network layers, including an input layer 11, an output layer 14 at least one internal layer 12, 13. The raw input speech data 1 from a speaker is encoded in an initial input representation Rᵢₙₚᵤₜ to allow it to be processed by the model. The input representation Rᵢₙₚᵤₜ is then input into the first layer 11 of the neural network and the model is trained to map the input representation Rᵢₙₚᵤₜ through the network layers 10 to an output representation Rₒᵤₜₚᵤₜ at the output layer 14 which is usable, by a prediction layer 15, for example a classification layer or a regression layer, to make a health condition prediction.

The input representation preferably comprises a feature vector, i.e. a vector encoding the input speech data into a format usable by the main model. At each layer of the neural network the received representation undergoes transformation by the application of the weights and activations at each node of the layer such that each layer 10 outputs a representation R, which is a transformed representation of the previous layer, to the subsequent network layer. By training the model to make a health condition prediction, the model 100 learns to adjust the parameters applied to the representations, such as the weights and activations, at each layer so that the input representation is progressively transformed through a series of internal representations to finally reach an output representation encoding information within the input speech data that is associated with the particular health condition and can be used to make the prediction.

In the example of FIG. 1, the model 100 includes a classification layer 15 which computes a number based on the output representation which can be thresholded to provide a binary decision, such as a positive or negative diagnosis of Alzheimer's. The main model is trained end to end with the classification layer using labelled speech data to map the input representation to the Alzheimer's diagnosis, such that each subsequent network layer 10 learns a further transformed representation of the input speech data 1 and, assuming the model is trained effectively on sufficient data, the output representation can be used to make a yes or no prediction to diagnose Alzheimer's. However, as described below, the health condition output may take a number of different forms and the trained model could be a pre-trained model which has only been trained on unlabelled data.

In prior art methods, generally the content of the representations of the internal network layers is unknown and the model is understood to be working effectively if it provides a reliable prediction based on labelled training data. However, it is often not certain why the model is making a certain prediction—which part of the complex information within the input speech data the model is using—and whether all of the rich information within the input speech is being utilised, particularly without any additional clinical understanding being provided to the model other than the target output.

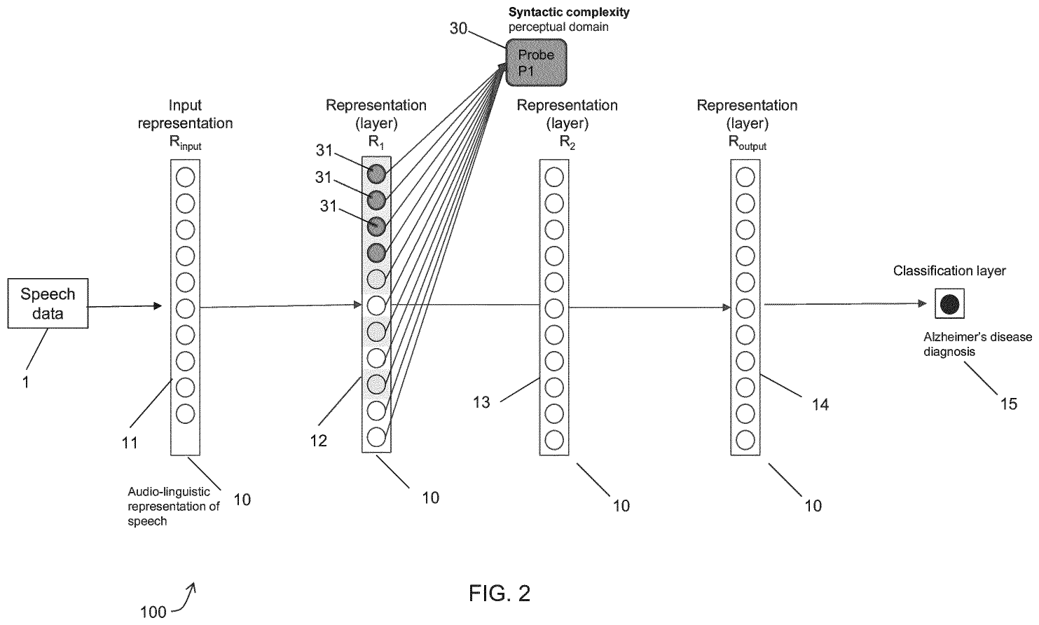

FIG. 2 illustrates a method according to the present invention for identifying the clinical information within the internal representations Rn of the internal network layers and to identify parts of these representations which could be used as representations for making further predictions. As schematically illustrated in FIG. 2, the method comprises training a probe model 30 independently of the main model 100, to map an internal representation R₁ of an internal network layer of the main model 100 to an independently determined measure of a clinically relevant feature of the input speech data 1 or the speaker, where a “clinically relevant feature” is a property of the speech or speaker which is impacted by a health condition and is determined independently of the main model—for example, a syntactic property such as the noun rate or a property of the speaker such as the speaker's score on a neuropsychological test.

The probe model 30 comprises a machine learning model, such as a simple classifier or regression model, which is trained in an adjacent task, separately to the main model, to map an internal representation Rn of an internal network layer 13 of the main model 100 to the clinically relevant feature of the input speech data 1 or the speaker. In some examples, as described below, a disentangling step may be performed on the internal representations with the probe model trained on the disentangled representations. In other examples, the main model may be configured to promote disengaging of representations, for example by appropriately configuring the loss function during training of the main model.

A separate probe model may be trained for each clinically relevant feature to which the internal representations are mapped. As described in more detail in the specific example below, the clinically relevant features are properties of the speech or speaker which are impacted by a heath condition and these may be grouped into “perceptual domains” which define groups of measures associated with a particular characteristic of the speech or speaker. Examples of domains include prosody, syntactic complexity and episodic memory.

In some examples, such as that illustrated in FIG. 2, one or more probe models 30 may be trained to map the internal representation to a domain vector comprising one or more clinically relevant features within a particular clinical domain associated with the condition. In the example of FIG. 2, where the main model provides an Alzheimer's diagnosis prediction, a probe may be trained for a number of clinical domains associated with Alzheimer's disease, for example a separate probe model could be trained for each of prosody, episodic memory and syntactic complexity. Each of these domain vectors then comprises one or more measures associated with that domain, where the measures may be objective automated measures of the input speech or human-rates measures. For example, as shown in FIG. 2, a syntactic complexity probe 30 may be trained to map an internal representation R₁ to a syntactic complexity domain vector. The syntactic complexity domain vector may comprise objective automated measures, such as the noun rate, the ratio of dependent clauses to T-units, mean length of clauses, number of verb phrases per T-unit etc., and human-rated measures such as a rating of syntactic complexity of the input speech.

By training the probe model 30 to predict the measures of the syntactic complexity from the internal representation R₁, the probe model learns which elements of the internal representation R₁ are the best predictors of the syntactic complexity vector. For example, the weights and activations that the probe P1 learns to apply to the representation R₁ can be used to determine which elements 31 of the representation R₁ are given most weight by the probe in determining a prediction of the syntactic complexity measures. It is these representation elements 31 that encode the most relevant information for predicting that particular clinically relevant feature.

The elements 31 identified by the probe P1 can be used to form a vector which encodes syntactic complexity information of the input speech. The main model may not rely on this syntactic complexity information to make its prediction but this information can now be fed into the main model to improve performance in making the health condition prediction.

This vector provides a new data representation for making a health condition prediction. For example it can be used to encode input speech data to provide an Alzheimer's diagnosis solely on the basis of (in this illustrative example) syntactic complexity. By forming these new data representations for a number of important clinical domains, the method allows a clinician to understand the influence of the different clinical domains on the health condition prediction to better diagnose a patient. In particular the method provides a more complete diagnosis since it provides a measure of the contribution to the overall Alzheimer's diagnosis by different domains. This provides more granular information on how a patient is affected by a particular health condition and so can be used to better diagnose patients, as well as builder better predictive models and to devise better treatment plans to focus on the particular domains most affected, as will be described.

### Main Model Structure and Training

The main model may be any neural network trained to map an input representation encoding speech data to an output representation for use in a health condition prediction. The speech data may include text and/or audio data of speech but preferably includes both the linguistic and acoustic content of a passage of speech. The input representation encodes linguistic, i.e. language features and/or acoustic speech information. Again, preferably the input representation encodes both linguistic and acoustic information to benefit from the full range of information available within the speech data.

In some examples the input representation may comprise selected features, extracted from the input speech. For example, features with known clinical rationale may be extracted from the input speech so as to impart additional clinical knowledge to the model. For example, given the noun rate is known to be an indicator for early Alzheimer's, the noun rate could be selected as an input feature within the input representation such that the main model does not have to learn this association during training.

In other preferable examples the main model may be a representation learning model, where features are not extracted manually but learnt in the process of training the model. An input representation, preferably comprising text and audio representations, is used to encode the raw speech data into a suitable format for processing and the model is trained to transform the input representation into an output representation which can be used by a prediction layer to provide a health condition prediction. By training the model end to end the model learns to transform the input representation into an appropriate output representation for providing the prediction of the health condition prediction.

Both feature based and representation learning models can be trained for use as the main model. Particularly advantageous model structures and training methods are described in the applicant's earlier European Patent Application number 20185364.2.

As described in the above mentioned patent application, the training may preferably take place in two stages. The first stage may comprise “pre-training” the model on large unlabelled data sets using unsupervised (or more specifically self-supervised) training in which one or parts of the input representation are masked or corrupted and the model is trained to predict the masked or corrupted representations, thereby learning internal representations which encode associations between the text and audio data usable to predict the masked audio or text representations. Given pre-training uses more widely available unlabelled speech data sets, it can be used to initialise the representations into a form which encodes general use information from the speech data which is usable in a subsequent health condition prediction.

The second stage may comprise task-specific fine tuning which the pre-trained model is fine-tuned using a smaller labelled data set for a particular health prediction task. Fine tuning involves encoding the labelled speech data into the input representation, adding a prediction layer 15 and training the model to map the input representation to the target health condition prediction such that the representations learnt by the model are further optimised for the particular heath prediction task.

After training of the main model, the model, and its representations, are frozen and no further changes to the model take place. The probe models are then trained using the fixed internal representations of the main model.

When the two stage training method of the main model is used, the probes may be trained on the pre-trained or fine-tuned model, although the methods of the present invention are preferably applied to the fine-tuned model to gain further information on the internal structure of the model relevant to the health condition prediction task of the fine-tuning step.

This two-stage training strategy is advantageous because it utilises more widely available non-labelled data sets to train the model and learn representations which encode information on the context of linguistic and acoustic features of language. The representations formed during this process therefore enclose a lot of general information on speech and language which can be utilised when fine tuning on the smaller clinical labelled data sets. However the fact that labelled clinical data sets are limited means that there is likely to be a large amount of useful information in the pre-trained representations which is not utilised by the main model when learning to make a health condition prediction during fine-tuning. The method of the present invention can be utilised to find associations within the data representations which are not being utilised by the main model to further improve its performance.

### Probe Model Structure and Training

The probe model may comprise any type of machine learning model which can be trained to predict a measure of a clinically relevant feature of the input speech or speaker based on a speech data representation. The probe preferably comprises a linear model, multi-layer perceptron, an attention-based model or a Bayesian neural network and is preferably simple such that it does not internally learn to do the task in a sophisticated way.

The probe is trained to predict the clinically relevant feature of the input speech using an internal representation encoding the input speech within an internal layer of the main model, thereby learning associations within the internal representations which might not be learnt by the main model.

The probe model can be used to identify elements 31 of an internal representation Rᵢₙₚᵤₜ which can be used to provide a prediction of the clinically relevant feature in a number of different ways. The elements can be selected based on those which provide the most accurate prediction or the elements can be selected based on those which require the simplest probe model structure or minimum amount of training data to provide a prediction of a given accuracy.

### Specific Example of a Method According to the Present Invention

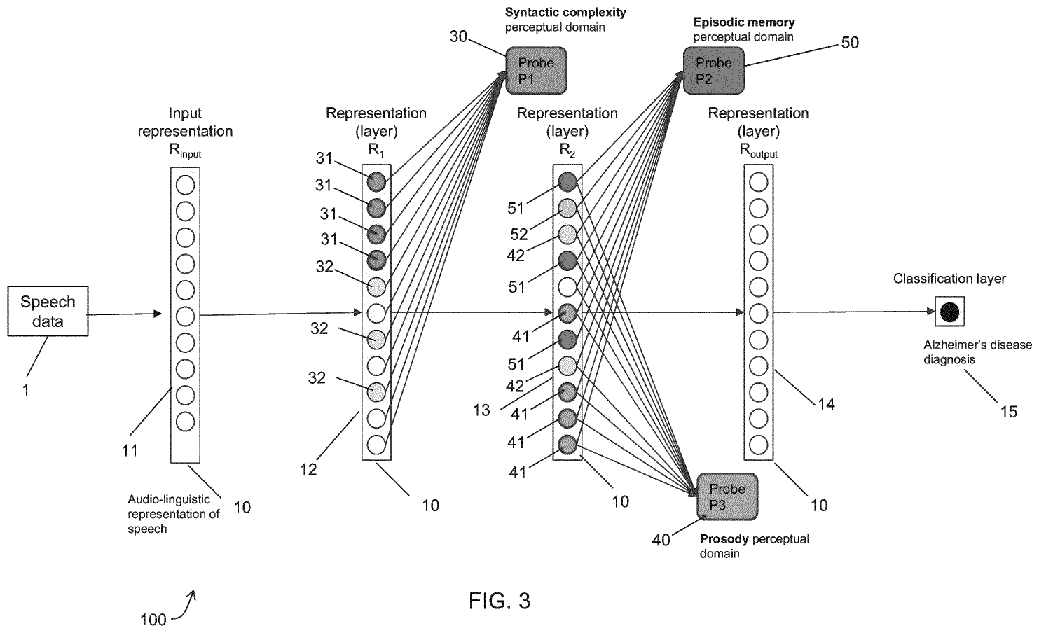

A specific example of the method according to the present invention is illustrated in FIG. 3.

Step 1: Train the predictive model on the primary classification/regression task using a neural architecture and freeze the layers.

As described above, a main model, comprising a neural network, is trained on a primary health condition prediction task. Again, for the purpose of this illustrative example, the task is an Alzheimer's diagnosis classification task, although it could be any predictive task for monitoring or diagnosis of a health condition which potentially causes detectable changes in the speech of a patient.

The raw speech data is encoded into the input representation Rᵢₙₚᵤₜ for processing. Preferably the input representation comprises audio representations encoding acoustic information of the raw speech data and linguistic representations encoding linguistic information of the input speech data. In certain preferable embodiments the input representations are combined audio-linguistic representation encoding the interrelation between the linguistic and acoustic information within the patient speech data. A method for forming such a combined audio-linguistic representation is described in European Patent Application number 20185364.2. In other examples the input representation might include solely audio, solely text or non-combined audio and text representations.

In this example, the model is trained on labelled speech data to predict the Alzheimer's diagnosis. Each subsequent layer learns a further transformed version of the input representation, with the final representation Rₒᵤₜₚᵤₜ of the output layer usable by the classification layer 15 to provide the diagnosis.

After training the layers 10 of the model are fixed and no further changes take place in the further steps of the method.

Step 2: Define a set of feature domains associated with the health condition.

These “perceptual domains” are characteristics of the speech or speaker which are related to the health condition. They should be as clinically meaningful, separable and comprehensive as possible.

Each domain relates to a characteristic of the speech of speaker, which is influenced by the health condition and can be measured or estimated in one or more ways. For example, for Alzheimer's disease the perceptual domains might include phonation, articulation, prosody, affect, memory and syntactic complexity. Each of these characteristics of the speech or speaker change in a patient with Alzheimer's disease and the associated information may or may not be learnt in the process of training the main model.

Step 3: For each perceptual domain, define one or more constituent features of the speech, within that domain, that can be measured or estimated.

The features may be objective measures of the input speech or they may be human-rated, possibly more subjective features. The objective measures, for example the noun rate, may be derived automatically from the input speech using automated speech recognition methods. Other features, such as the human-rated scores may need to be assessed independently so that the training data set includes these measures of the speaker or speech.

For example in the case of the syntactic complexity domain, the objective automated measures of the speech may include the noun rate, the ratio of dependent clauses to T-units, mean length of clauses, number of verb phrases per T-unit etc., all of which may be derived automatically from the input audio and/or text data. The human-rated measures of syntactic complexity may include a human rating of syntactic complexity of the input speech, which would need to be assessed independently.

In contrast, for the episodic memory domain, the measures are generally carried out by way of neuropsychological test on the speaker, for example to provide a score for verbal episodic memory and a score for visual episodic memory.

Step 4: Apply one probe model to every layer in the trained main model for every feature in every perceptual domain and train all probes independently.

The probe model comprises a machine learning model but may take a number of different forms. Preferably it is a simple linear classifier or regression model, or an attention-based model. The probe models are simple models such that the probe cannot learn to do the task in a sophisticated way but instead simply learns the elements of the internal representation that can be used to predict the clinically relevant feature.

The probe models may be trained on the same speech data set used to train the primary prediction task of the main model or on a separate speech data set. The model training data is fed into the main model to get the internal representations of the training data and each probe is trained to predict the corresponding measure of the clinically relevant feature of the training data from the internal representations of the network layer to which it is applied.

The illustrative example of FIG. 3 shows a first probe 30 trained to map the internal representation R₁ of a first internal network layer 12 to the measures of syntactic complexity of the input speech data, a second probe 50 trained to map the internal representation R₂ of a second internal network layer 13 to the measures of Episodic memory of the speaker, and a third probe 40 trained to map the same internal representation R₂ of the second internal network layer 13 to the measures of the prosody of the input speech data.

Step 5: For each perceptual domain, find the layer at which its features overall can be predicted the best and in the most disentangled way.

For each domain, a separate probe is trained to predict the constituent features of that domain based on the internal representations of each network layer The one or more internal network layers may be selected based on one or more of (1) the accuracy of the prediction of the clinical feature provided by the internal representation of the layer; (2) the disentanglement of the elements of the layer; (3) the “amount of effort” required by the probe to achieve the prediction, in particular (i) the size of a probing model, and/or (ii) the amount of data needed to achieve a required prediction accuracy.

The representations are disentangled where certain elements of the representation decouple from the remaining elements and contribute much more strongly to the prediction of the clinically relevant feature. In this situation the clinical information usable to provide the prediction is encoded in a selection of well-defined sub-elements of the representations.

Step 6: Use the internal parameters learnt by each probe model to identify elements of the probed representation that are being used by the probe to predict the domain features.

During training of the probe model, the probe model adjusts various internal parameters in order to learn how to map the internal representation to the feature value. The internal parameters of the probe may include neuron weights, biases and activations. For example, the probe may be a simple neural network which learns the magnitude of the weight to apply to each element of the representation in order to provide the best prediction of the corresponding feature of the input speech. The learnt weights therefore indicate the elements of the representation which encode the most relevant information usable by the probe in making the prediction.

The probe weights can be thresholded to define the significance level at which the representation elements should be identified as being linked to the corresponding clinical domain probed by the probe model.

As illustrated in FIG. 3, the trained syntactic complexity domain probe 30 has identified representation elements 31 and 32 in the first internal network layer 12 as encoding information predictive for syntactic complexity, the trained episodic memory probe P2 has identified representation elements 51 and 52 in the second internal network layer 13 as being predictive for the episodic memory domain features and the prosody domain probe P3 has identified elements 41 and 42 as encoding information in the input speech usable for predicting the prosody domain features. However the representation elements 31, 41 and 51 make a much stronger contribution to the prediction than representation elements 32, 42 and 52 and therefore the corresponding probe learns to apply greater weight to these elements.

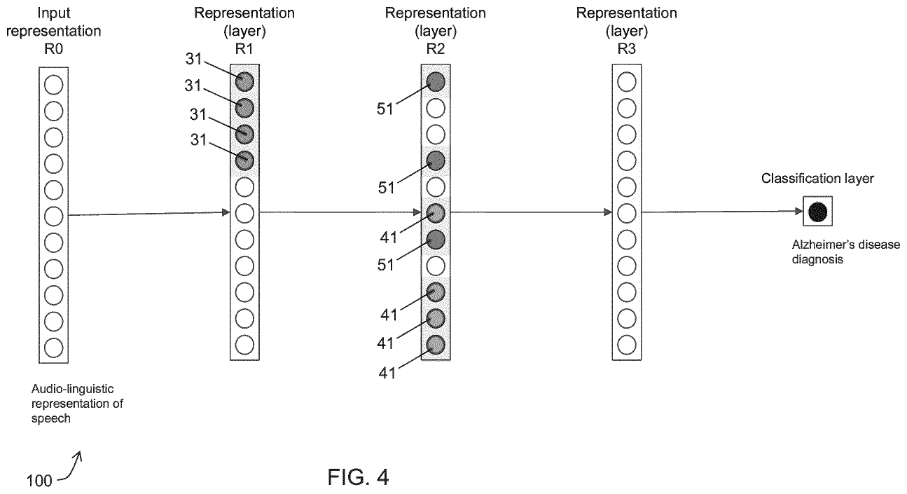

The probe weights (and/or activations) are therefore thresholded to select only those representation elements which provide the most significant contribution to the prediction, as determined by the selected threshold. As shown in FIG. 4, only the representation elements 31, 41, 52 within the network layer which make the strongest contribution remain after thresholding.

Therefore after training the probes, a set of representation elements or “features” of the input speech is identified for each domain. The representation elements for each domain may come from a single layer or may be selected from multiple layers, where individual elements across layers are found to best predict the domain features. In some examples, certain vector elements may be shared between domains.

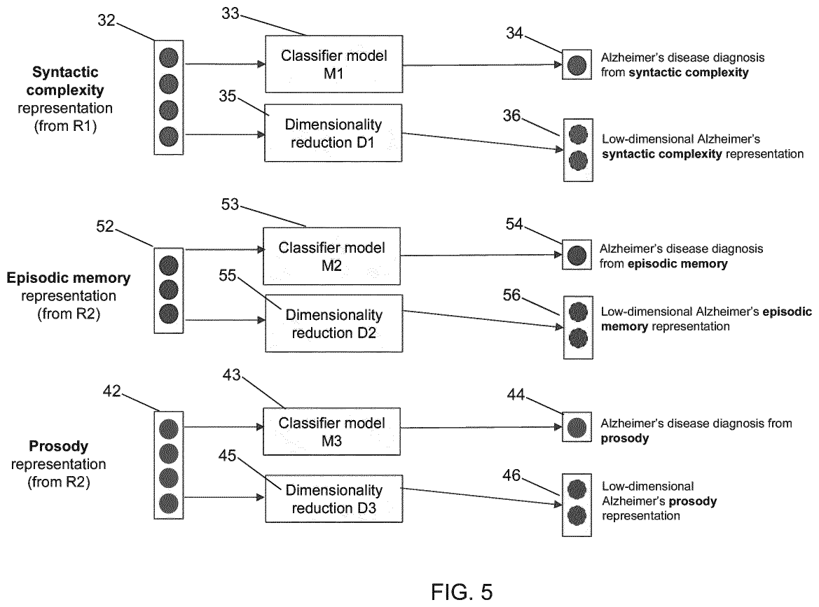

Each set of representation elements corresponding to a particular domain may be extracted from the network and combined into a domain vector. As shown in FIG. 5 the output of the illustrated exemplary method is a syntactic complexity domain vector 32, an episodic memory domain vector 52 and a prosody domain vector 42.

These domain vectors 32, 42, 52 output from the method may be used in a number of ways. Importantly they provide information on the impact of that domain in the main model reaching the health condition prediction, in this case Alzheimer's, but they also provide data representations which can be used to encode input speech data for use in a new model, imparting greater clinical understanding into a predictive model and reducing the learning that the model must do, allowing for improved predictive performance with smaller data sets, as explained further below.

The following optional steps illustrate how the domain vectors can be used to perform a number of additional tasks and provide additional outputs.

Step 7: Perform a prediction on the main task using the perceptual domain vector.

As shown in FIG. 5, the domain vectors 32, 42, 52 can be input into a corresponding classifier model 33, 43, 53 to provide a health condition prediction corresponding to that provided by the main model. For example, where the main task was an Alzheimer's prediction, the domain vectors can be used to encode speech data to be input into a new, arbitrarily complex, model for making an Alzheimer's diagnosis. For example the syntactic complexity domain vector 32 can be input into a classifier model 33 to output an Alzheimer's disease diagnosis 34 based solely on syntactic complexity. This output provides a task relevant score for that domain, for example the probability that the speaker has Alzheimer's based only on syntactic complexity.

By inputting each of the domain vectors into a corresponding classifier it is possible to determine a component of the diagnosis corresponding to each domain. This set of scores ‘explains’ the overall Alzheimer's diagnosis, providing information on which aspects of the input speech are most indicative of an Alzheimer's diagnosis. This information is of significant value in both better understanding a particular health condition, the symptoms and how it effects speech. This output can also be used to help inform the building of better, more accurate predictive models.

As explained above, each domain vector also forms a newly identified data representation 32, 42, 52 for input speech that can be used as additional input to a diagnostic model.

Step 8: Form lower-dimensional representations of the domain vectors.

Alternatively or additionally, dimensionality reduction 35, 45, 55 may be performed on the domain vectors 32, 42, 52 to provide a reduced dimension domain vector 36, 46, 56. These can be used as the input to a classification or regression model, reducing the computational requirement in order to provide a diagnosis. This can also preserve more general information and help create a disentangled, potentially de-identified representations of the input speech.

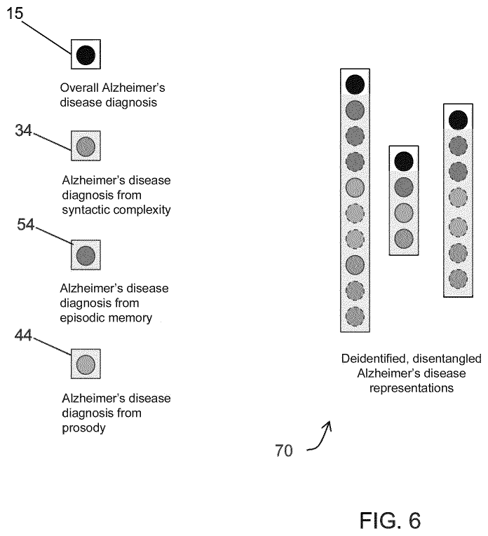

The output products of the method after performing the additional steps 7 and 8 are shown in FIG. 6. The products of the method include: the original Alzheimer's diagnosis 15 output by the main model and the Alzheimer's disease diagnosis based solely on the domain vectors for each of syntactic complexity 34, episodic memory 54 and prosody 44, each giving a measure of the contribution of this speech or speaker characteristic on the overall Alzheimer's diagnosis.

These products provide a diagnostic kit that can be utilised by a clinician to provide a more accurate and complete Alzheimer's diagnosis. In particular the contribution of the different domains to the overall diagnosis could inform the clinician as to how advanced the Alzheimer's disease is, and indicate the severity of different symptoms to understand the ways in which it is affecting the patient. This understanding, and the more complete picture of the effects of the disease on a particular patient, can inform the treatment and care plan.

The output products shown in FIG. 6 also include a set of vectors 70 comprising the representations of the speech data that were used for the diagnosis. In particular the vectors comprise the original representation elements 31, 41, 51, identified by the probe models, possibly after additional dimension reduction. These vectors 70 therefore provide new disentangled speech data representations usable to provide an improved Alzheimer's diagnosis with a more complete picture of the contribution of the various domains.

If enough perceptual domains are used, which together provide sufficient granularity, the vectors formed from the identified speech data representations can replace the general speech data representations used as the input representation Rᵢₙₚᵤₜ. That is, patient speech to be tested can be encoded directly into the “combined domain vector” (formed from the representation elements identified by each probe) and this can be used as the input into a predictive model to provide a health condition prediction.

Using a vector formed by the domain probes in this way has a number of advantages. Firstly, it can provide a reduced dimension representation compared to a general speech representation, reducing the computation requirement for training. The vectors can thus provide more efficient data representations which encode just the relevant clinical domain data for making a particular diagnosis. This shares the advantages of feature based methods in which features are extracted and placed into a vector but provides additional advantages in that it utilises the work of the main model in pre-forming more compact data representations during training of the main task, and also allows for a wider range of clinically relevant features to be used, including features of the speaker and human-based ratings such as neuropsychological tests.

Importantly, the vectors prepared from the representation elements identified by the domain probes can be prepared such that they are de-identified from the original speaker. In particular, the vectors prepared using probes for predicting clinical features in this way can select representations which are invariant to nuisance variables such as speaker gender, age or identity. Therefore the method can provide speech data representations which are de-identified from the original speaker. De-identified representations are particularly desirable as they mean patient data can be anonymised prior to testing to meet patient data privacy regulations.

By encoding patient speech data in the de-identified vectors formed by the domain probes, patient data can be stored for analysis in anonymised form, unlike general speech data representations from which the speaker identity can be determined.

### Additional Disentanglement Steps

In certain preferable examples of the method according to the present invention, additional steps may be provided as part of the probing process to improve disentanglement of the internal representations of the main model. In particular, ideally the probe will identify a small number of representation elements which decouple from the remaining elements to encode the majority of the information relevant to a particular domain. In this way, a compact domain vector may be formed of relatively few elements which encode the vast majority of the relevant information to predict the features of that domain. To further promote the learning of disentangled representations, one or more additional steps may be taken.

A first option is to improve disentanglement of the representations learned by the main model by adapting the model structure and/or training strategy. In particular the loss function used in training the main model may be adapted to promote the learning of disentangled representations. For example the model may be a beta-VAE model as described in Higgins, I. et al. “beta-VAE: Learning Basic Visual Concepts with a Constrained Variational Framework.” ICLR (2017). In this way, the trained main model will have sufficiently disentangled internal representations.

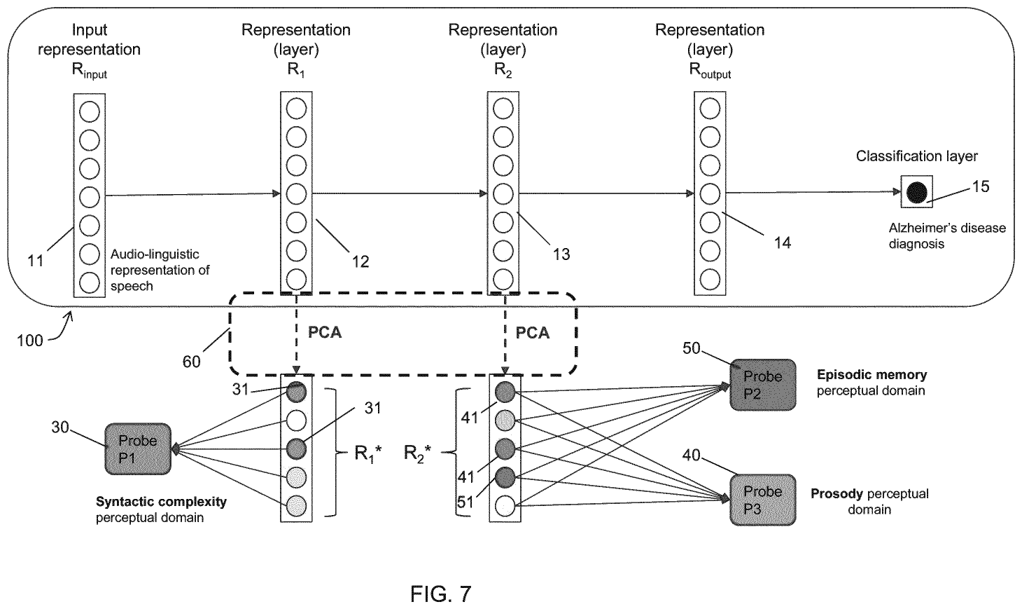

A second option is to carry out an additional intermediate step to perform disentanglement on the internal representations prior to application of the probe models, as illustrated in FIG. 7. In particular a disentanglement step 60 is applied to the internal representations R₁, R₂ of the trained main model 100. For example, principal component analysis may be performed on each internal representation R₁, R₂ to form a corresponding disentangled representation R₁*, R₂*, as shown in FIG. 7. This method proceeds exactly as that described above, with the main model 100 first trained on a primary prediction task. As above, in the example of FIG. 7 this is an Alzheimer's prediction task, although it could be any health condition prediction.

After training of the main model the layers are their constituent representations are fixed. Input speech data is input into the fixed main model to encode the speech data into the internal representations, R₁ and R₂. A principal component analysis (PCA) is then performed on the elements of each internal representation to form a corresponding disentangled representation R₁*, R₂*, formed of a smaller number of elements. This method helps enhance disentanglement of the representations such that the information for a particular domain is encoded predominantly in a smaller number of representation elements in a transformed, reduced dimension vector space. Performing PCA on the representation elements of the internal representations therefore promotes the formation of a reduced number of disentangled vector elements, which form the disentangled representations R₁*, R₂*.

The method then continues as above from Step 4, with each probe model applied to predict the clinically relevant feature from the disentangled representations R₁*, R₂*. As before the probe model learns which of the elements 31, 41, 51 of the disentangled representations R₁*, R₂* that provide the most accurate prediction of the feature and these are selected to form the domain vectors.

In examples incorporating a disentanglement step, such as PCA, the disentangling step is considered part of the probe. In other words the step “training a probe comprising a machine learning model to map an internal representation . . . ” comprises (1) performing disentanglement on the internal representation to provide a disentangled representation and (2) training the machine learning model of the probe to the disentangled representation to map the disentangled representation to the independently determined measure of a clinically relevant feature associated with a particular domain.

As before the elements 31, 41, 51 of the disentangled representations R₁*, R₂* may be selected by the probe based on the weights and/or activation learnt by the probe model. As above, the internal network layers may be selected based on one or more of (1) the accuracy of the prediction of the clinical feature provided by the internal representation of the layer; (2) the disentanglement of the elements of the layer; (3) the “amount of effort” required by the probe to achieve the prediction, in particular (i) the size of a probing model, and/or (ii) the amount of data needed to achieve the high quality.

Quantifying the Relevant Information Encoded within the Representations

Furthermore, and importantly, probing can provide a quantifiable measure of the success of predicting a particular clinically relevant feature. Therefore when the method is applied in a providing a health condition application, this quantifiable probing technique can provide a quantified measure of the internal representations' success in encoding the relevant speech or speaker property, which can be provided as an output to a user.

To quantify how well the trained representations encode the clinically relevant speech signals, the method may use the accuracy of the probe or more preferably it may employ information-theoretic probing with minimum description length (as described in “Information-Theoretic Probing with Minimum Description Length”, Proceedings of the 2020 Conference on Empirical Methods in Natural Language Processing, pages 183-196, Nov. 16-20, 2020). This technique provides an objective measure of how well information is encoded in the representations for each of the clinically relevant features predictive. In particular, it gives a measure of either (i) the size of a probing model or (ii) the amount of data needed to achieve a particular prediction accuracy.

### Specific Application for Audio Representations

One important application of the present invention is the application of the method to probe audio representations, in particular prosody representations, which are particularly strong representations for use in health condition prediction tasks.

Prosody refers to the non-linguistic content of speech. Prosody is often defined substractively, for example as “the variation in speech signals that remains after accounting for variation due to phonetics, speaker identity, and channel effects (i.e. the recording environment)”. It can also be defined as the combination of timbre of speech (the spectral information which characterises a particular voice), the rhythm, pitch and tempo. Tempo relates to the speed and duration of voiced segments, while rhythm relates to the stress and intonation.

There are a large range of diseases that impinge upon the correct functioning of these physiological systems resulting in changes to both choice of language but also non-linguistic components, for example the hesitations, pitch, tempo and rhythm. For example cognitive disorders such as Alzheimer's affect the brain and therefore impact on speech through both the higher-level speech systems such as memory but also the lower-level physiology in terms of the brain's ability to control the vocal cord and articulatory system. Therefore there is a particular need for obtaining strong prosodic representations for use in speech analysis for health condition predictions. One significant issue is the difficulty in extracting prosodic representations which retain expressivity and encode all of the important non-linguistic information necessary for downstream speech analysis tasks, while being sufficiently de-identified from the speaker to protect user privacy and meet GDPR/HIPAA requirements. Much of the non-linguistic components of speech overlap with signals in the speech which are characteristic of the speaker.

Therefore the probing methods of the present invention can be applied to prosody representations to determine the extent to which the identifying (timbral) information has been removed and just the required non-timbral prosody components remain—which are those required for making strong health condition predictions. In particular the main model may be a model for encoding speech in prosody representations and the method of the invention may be applied by training a probe comprising a machine learning model, independently to the training of the main model, to map a prosodic representation of the input speech data to an independently determined measure of a clinically relevant feature of the input speech data or the speaker.

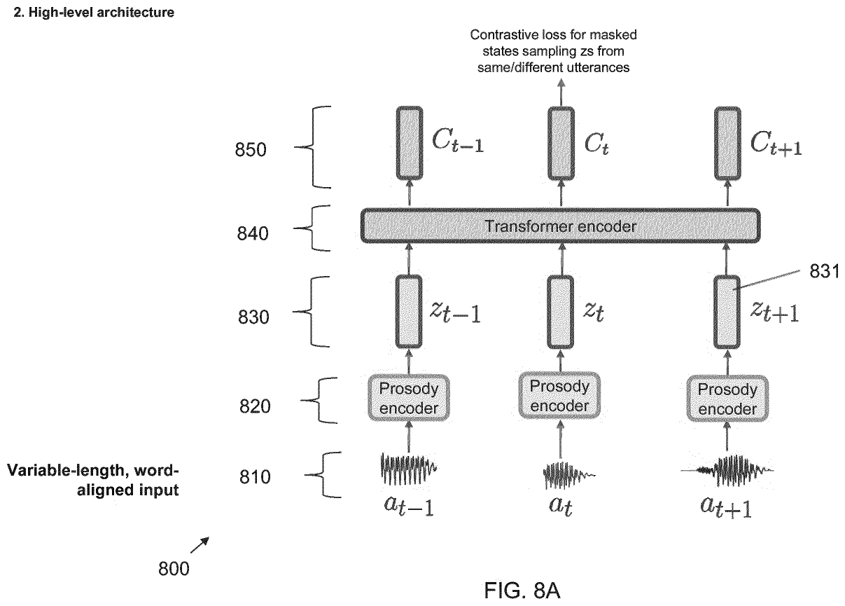

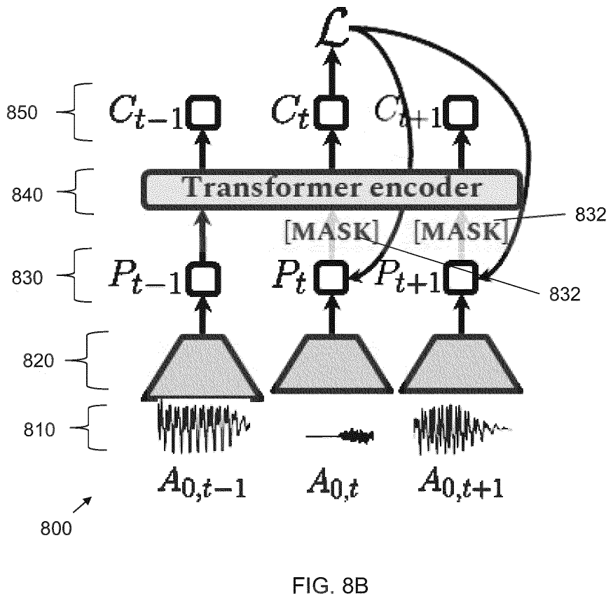

FIGS. 8A and 8B illustrate a possibility for a machine learning model for encoding prosodic representations.

### Overview of Example Encoder Model Architecture

The prosody encoder model may be any model suitable for encoding the pre-processed sections of audio data into quantised audio representations. The prosody encoder preferably includes a machine learning model, trained to map sections of processed audio data to corresponding quantised audio representations of the sections of audio data.

FIG. 8 schematically illustrates a high-level view of an example of a possible prosody encoder model 800.

The input 810 to the model is sections of the pre-processed audio data. Preferably this comprises variable length, word-aligned audio, i.e. sections of the processed audio data which each include one spoken word. These sections of processed data are referred to as “audio words”.

The first stage of the model is the prosody encoder 820. This is a model, or series of models, configured to take one audio word as input and encode this single word as a corresponding quantised audio representation encoding the prosodic information of the audio word. Prosodic information is effectively encoded due to the pre-processing to remove speaker-identifying information from the raw audio input, in particular timbre, and due to various features of the model, described in more detail below.

The output of the prosody encoder stage 820 is therefore a sequence of quantised prosody representations 830, each encoding the prosodic information of one spoken word within the input speech and therefore together in sequence encoding the prosodic information of a length of audio data.

The prosody encoder 820 may have several possible different structures. As described below, in one example the prosody encoder comprises a first stage configured to encode each input audio word as a non-quantised audio representation and a second stage configured to quantise each non-quantised audio representations into one of a fixed number of quantised prosodic states (quantised prosody representations or prosody tokens). Further possible implementation details of the prosody encoder are set out below.

The sequence of prosody tokens 830 is then fed into a contexualiser model 840 to encode the quantised prosody representations into contexualised prosody representations. The contextualisation model 840 is preferably a sequence-to-sequence machine learning model configured to encode contextual information of a particular prosody token 831 into a new representation. The model is configured to encode information about the relationships between a quantised prosody representation 831 and the surrounding quantised representations within the sequence 830—commonly referred to as “context”. The contextualisation model 840 is preferably an attention based model, in particular a transformer encoder.

The output of the contextualisation model 840 is a sequence of contextualised prosody representations 850, each encoding the prosodic information of a particular audio word in the sequence and its relationship to the surrounding prosodic information in the sequence.

Both the tokenized prosody representations 830 or the contextualized prosody representations 850 can be used for downstream tasks, like expressive text-to-speech systems, spoken language understanding and speech analysis for the monitoring and diagnosis of a health condition. Both sets of representations encode just the prosodic information of the speech and are substantially de-identified so may be used where anonymising of user data is required.

### Overview of Model Training

FIG. 8B schematically illustrates a method of training an encoder model of FIG. 8A for use in the method according to the present invention.

Firstly the pre-processing is carried out on a training data set comprising raw audio speech data. The pre-processed raw audio 810 is fed into the prosody encoder 820, which produces one set of prosody tokens (P_i) 830 for each audio-word 810. In the illustrated example there are 3 tokens for each audio-word 810 but it can be 1 or more. At this stage, the model is completely non-contextual—each representation has only ever seen the audio for its own audio-word and not any information from the surrounding parts of the audio data. As described above the mode then comprises a contexulisation encoder 840, preferably a transformer, configured to encode the prosody tokens into contextualised representations 850.

The training process used is a form of self-supervised learning in which the model is trained to predict masked tokens from the surrounding context. This is a similar approach to that used in masked language models (see for example “BERT: Pre-training of Deep Bidirectional Transformers for Language Understanding”, Devlin et al. arXiv:1810.04805) but in this case the model uses solely audio, prosodic information and instead of training the model to predict the masked token a contrastive training approach is used in which the model is trained to predict the correct token from a number of different tokens.

In more detail, one or more tokens 830 output by the prosody encoder 820 are randomly masked 832, the model is given a number, for example 10, possible tokens and the model is then trained to predict the correct one from the group of possible tokens (i.e. which token corresponds to the token that has been masked). The other 9 tokens are masked states from other masked audio-words. One preferable feature of the training process is that the other tokens (the negatives) are selected from the same speaker. In this way the model is not encouraged to encode information that helps separates speakers and therefore further aids de-identification of the representations.

The network 800 is trained end to end so the prosody encoder 820 is trained together with the transformer encoder 840.

Preferably the model is configured to learn to always represent prosody as the same token at every timestep—so that the contextual prediction can be done with 100% accuracy. Once trained, input speech data can be fed into the model and either or both of the contextual representations (post-Transformer) or the pre-Transformer non-contextualized representations (or from any layer inside the Transformer) can be used for downstream speech processing tasks.

### Application of the Probe Model

A probe model may then be applied as described above, with the probe trained, independently to the training of the encoder, to map a prosody representation to an independently determined measure of a clinically relevant feature of the input speech data or the speaker, where a clinically relevant feature is a property of the input speech or speaker that is impacted by a health condition.

By examining the success of the model in predicting a component of prosody it can be determined to what extent the prosodic representations encode information in speech related to that component. Furthermore, and importantly, probing can provide a quantifiable measure of the success of predicting a particular measure of prosody. Therefore when the method is applied in a technical application, this quantifiable probing technique, can provide a quantified measure of the prosodic representations' success in encoding the relevant prosodic property, which can be provided as an output to a user.

Of particular relevance is confirming that the prosodic representations encode each of the required components of prosody, other than the speaker identifying characteristics—timbre in particular. Therefore the method may further comprise training a probe model to predict audio features representative of the subcomponents of prosody: pitch, rhythm, tempo and timbre.

For pitch a probe model may be trained to predict the median pitch. For rhythm probe models may be trained to predict median word intensity and number of syllables. For tempo, probe models may be trained to predict articulation rate (syllables per second), speech rate, average syllable duration, and word duration (including pre-silence). For timbre, probe models may be trained to predict the median formants F1, F2, F3 (shifted).

To quantify how well the trained representations encode the prosodic information, the method may use the accuracy of the probe or more preferably it may employ information-theoretic probing with minimum description length (as described in “Information-Theoretic Probing with Minimum Description Length”, Proceedings of the 2020 Conference on Empirical Methods in Natural Language Processing, pages 183-196, Nov. 16-20, 2020). This technique provides an objective measure of how well information is encoded in the quantised audio representations for each of the audio features representative of each subcomponent of prosody.

The probe models may be applied to both the quantised prosodic representations output from the product quantiser and the contextualised prosodic representations output from the contextualisation model, to provide an output to a user to inform on the information that is being encoded. The probe models may also be applied to the components of to product quantizer, where the product quantizer forms part of the prosody encoder and is configured to quantise the non-quantised representations provided by an initial encoding layer into a number of prosody components, preferably three. The application of the latter has shown that a product quantizer has the ability to naturally disentangle the information into the three non-timbral components of prosody.

The probe models comprise a machine learning model, preferably a simple classifier or regression model, trained separately to the encoder models to map one or more audio representations provided by the model to a measure of prosody. The probe preferably comprises a linear model, multi-layer perceptron, an attention-based model or a Bayesian neural network and is preferably simple such that it does not internally learn to do the task in a sophisticated way.

1. A computer-implemented method for identifying speech data representations for monitoring or diagnosis of a health condition, the method comprising:
providing a main model comprising a trained neural network, trained to map an input representation encoding input speech data to an output representation for use in providing a health condition prediction, the neural network comprising one or more internal network layers each comprising a representation of the speech data which is passed to a subsequent network layer of the neural network, where the representations of the internal network layers are referred to as internal representations of the trained neural network;
inputting speech data from a speaker into the main model to form the internal representations of the input speech data; and
training a probe comprising a machine learning model, independently to the training of the main model, to map an internal representation of the input speech data to a measure of a clinically relevant feature of the input speech data or the speaker, where a clinically relevant feature is a property of the input speech or speaker that is impacted by a health condition.

2. The computer-implemented method of claim 1 wherein training the probe model independently to training of the main model comprises:
fixing the main model after training and, in a separate training task, training the probe to map a fixed internal representation of the input speech data to the independently determined measure of a clinically relevant feature of the input speech data or the speaker.

3. The computer-implemented method of claim 1 wherein the main model is trained to map an input representation encoding input speech data to a health condition prediction.

4. The computer-implemented method of claim 1 wherein the measure of the clinically relevant feature of the input speech data or the speaker is determined independently of the main model.

5. The computer-implemented method of claim 1 comprising using the trained probe model to identify elements of the internal representation that:
encode more information usable by the probe for predicting the clinically relevant feature relative to the remaining elements of the representation or other internal representations; and/or
decouple from the remaining elements of the internal representation in predicting the clinically relevant feature.

6. The computer-implemented method of claim 5 wherein the elements of the internal representation are identified according to parameters of the machine learning model of the probe learnt during training, wherein the parameters preferably comprise one or more of weights, biases and activations learnt by the machine learning model of the probe.

7. The computer-implemented method of claim 5 wherein the identified elements are used to form speech data representations which are invariant to one or more of:
speaker identity, speaker age, speaker gender.

8. The computer-implemented method of claim 5 wherein the main model has a plurality of internal network layers, the method comprising:
training a probe for each of a plurality of the internal network layers to map the corresponding internal representation to the measure of the clinically relevant feature of the input speech data; and
selecting one or more layers according to one or more of: (1) the accuracy of the prediction of the clinically relevant feature provided by the internal representation of the layer; (2) the degree to which certain elements of the layer decouple from remaining elements of the layer in making the prediction; (3) the size or complexity of the probe model required to provide a given prediction accuracy, (4) the amount of input speech data needed to train the probe to achieve a given prediction accuracy; and (5) minimum amount of data per example to perform the task.

9. The computer-implemented method of claim 5 wherein the main model comprises
a supervised, unsupervised, self-supervised or semi-supervised model for making
a health condition prediction, the method further comprising:
inputting the identified elements of the internal representation into a machine learning model to determine a prediction of the health condition based solely on the identified elements associated with the clinically relevant feature.

10. The computer-implemented method of claim 1 wherein the probe comprises a linear model, multi-layer perceptron, an attention-based model or a Bayesian neural network.

11. The computer-implemented method of claim 1 wherein the method comprises:
fixing the main model once trained; and
subsequently training the probe model to map an internal representation of an internal network layer of the fixed main model to the independently determined measure of a clinically relevant feature.

12. The computer-implemented method of claim 1 wherein training the probe comprises:
performing a principal components analysis on the internal representation of an internal network layer to provide a disentangled internal representation; and
training the machine learning model of the probe to map the disentangled internal representation to the independently determined measure of a clinically relevant feature.

13. The computer-implemented method of claim 1 wherein the clinically relevant feature comprises one or more of:
an objective property of the input speech, preferably a phonological, prosodic, lexico-semantic or syntactic property;
a property of the speaker, preferably the speaker's score on a neuropsychological test; or
a clinician's rating of the speech or speaker.

14. The computer-implemented method of claim 1 wherein providing the trained main model comprises:
pre-training the main model, preferably using an unsupervised learning task on an unlabelled training data set; and
performing task specific training on the pre-trained main model using a second training data set with labels associated with a specific health monitoring or diagnosis task, to provide the trained main model.

15. The computer-implemented method of claim 1 wherein the main model is trained using a loss function configured so as to encourage the model to learn disentangled internal representations.

16. The computer-implemented of claim 1 wherein the main model comprises a classifier or regression model trained to provide a health condition prediction based on the input representation of the input speech data, the method comprising:
obtaining a measure of a plurality of clinically relevant features, each clinically relevant feature comprising a property of the speech or speaker which is impacted by the health condition predicted by the main model; and for each clinically relevant feature:
applying a separate probe to each of a plurality of the internal network layers of the main model, and training all probes independently to map the corresponding internal representation to the measure of the clinically relevant feature;
identifying one or more network layers by training a probe for each of a plurality of the internal network layers to map the corresponding internal representation to the measure of the clinically relevant feature of the input speech data and selecting one or more layers according to one or more of: (1) the accuracy of the prediction of the clinically relevant feature provided by the internal representation of the layer; (2) the degree to which certain elements of the layer decouple from remaining elements of the layer in making the prediction; (3) the size or complexity of the probe model required to provide a given prediction accuracy, (4) the amount of input speech data needed to train the probe to achieve a given prediction accuracy; and (5) minimum amount of data per example to perform the task;
selecting elements of the corresponding internal representations of the selected network layers by encoding more information usable by the probe for predicting the clinically relevant feature relative to the remaining elements of the representation or other internal representations and/or decoupling from the remaining elements of the internal representation in predicting the clinically relevant feature; and
combining the selected elements into one or more vectors.

17. The computer-implemented method of claim 16 further comprising:
encoding input speech data into the one or more vectors; and
inputting the vectors into the main model or another machine learning model to provide a health condition prediction.

18. The computer-implemented method of claim 1 wherein the health condition is related to one or more of a cognitive or neurodegenerative disease, motor disorder, affective disorder, neurobehavioral condition, head injury or stroke.
